(19) United States
(12) Patent Application Publication
(10) Pub. No.: US 2015/0315544 A1
(43) Pub. Date: Nov. 5, 2015

(54) BLOOD SEPARATION FILTER

(71) Applicant: BIOMET MANUFACTURING, LLC, Warsaw, IN (US)

(72) Inventor: Ned Hamman, Leesburg, IN (US)

(73) Assignee: BIOMET MANUFACTURING, LLC, Warsaw, IN (US)

(21) Appl. No.: 14/269,478

(22) Filed: May 5, 2014

(51) Int. Cl. C12N 5/078

(52) U.S. Cl. CPC C12N 5/0641

(57) ABSTRACT

A filter for separating blood components includes a first pleated wall having a collection end and a second pleated wall. The second pleated wall converges with the first pleated wall at the collection end. The first pleated wall and second pleated wall are both composed of a material that is porous and configured as a barrier to red blood cells.

### BACKGROUND OF THE INVENTION

1. Field of the Invention

The present invention relates to blood separation devices, and, more particularly, to blood separation filters.

2. Description of the Related Art

Whole blood is a mixture of many different components including red blood cells (RBCs), white blood cells (WBCs), plasma, and platelets. It is often desirable to separate the components of whole blood so that they can be stored and transported in a more convenient manner. Whole blood is typically collected from a donor and then processed off-site to test the blood for any diseases, separate the components of the whole blood and get the individual components prepared for storage and transport. The separated blood components can then be transfused into patients who may or may not be in need of all the components found in whole blood.

One unique aspect of RBCs is that, unlike most cells in the body, they do not have associated mitochondria to produce adenine triphosphate (ATP), which acts as an energy source for cellular reactions. When RBCs are stored for a prolonged period of time, ATP concentrations in the stored RBCs can drop, which leads to decreased cellular activity. ATP also signals endothelial cells to release nitric oxide, which is a vasodilator. In addition to ATP levels dropping in stored RBCs, the concentration of 2,3-diphosphoglycerate (2,3-DPG) significantly drops after 14 days of storage. 2,3-DPG acts to decrease the binding affinity of hemoglobin to oxygen, making the RBCs more effective at releasing associated oxygen to surrounding tissues. The drops in ATP and 2,3-DPG make RBCs that have been stored for more than 14 days significantly less effective at oxygenating tissue than in vivo RBCs.

To counteract the effect of long storage periods on RBCs, techniques have been developed to “rejuvenate” the RBCs so that ATP and 2,3-DPG levels are not significantly decreased during storage. One such technique used is to mix the RBCs with a rejuvenating solution, such as rejuvesol® Red Blood Cell Processing Solution (rejuvesol® Solution), which has been marketed by Cytosol Laboratories Inc. (now Citra Labs, LLC) since 1991. Rejuvesol® Solution is a mixture that contains sodium pyruvate, inosine, adenine, dibasic sodium phosphate and monobasic sodium phosphate and has been found to maintain the levels of ATP and 2,3-DPG in stored RBCs.

Once the rejuvesol® Solution is added to the RBCs, it is removed from the RBCs prior to transfusion into a patient, which is referred to as “washing” the rejuvenated RBCs. However, not all separation techniques are appropriate to wash the rejuvenated RBCs, due to the cells' frailty.

What is needed in the art is a separation technique that can separate blood components from a blood solution.

### SUMMARY OF THE INVENTION

The present invention provides a pleated filter that can separate red blood cells from a rejuvenated blood product or other blood solution.

The invention in one form is directed to a filter for separating blood components that includes a first pleated wall with a collection end and a second pleated wall. The second pleated wall converges with the first pleated wall at the collection end. The first pleated wall and second pleated wall are both composed of a material that is porous and configured as a barrier to red blood cells.

The invention in another form is directed to a blood separation device that includes a vessel, a blood inlet formed in the vessel and a pleated filter placed within the vessel. The pleated filter separates the vessel into an inlet chamber that is fluidly connected to the blood inlet and a filtrate chamber. The pleated filter is configured as a red blood cell barrier between the inlet chamber and the filtrate chamber.

The invention to yet another form is directed to a method of collecting red blood cells that includes the step of providing a rejuvenated blood product that includes red bloods cells and a rejuvenating solution. The rejuvenated blood product is mixed with a wash solution to form a washed blood solution. A pleated filter is provided that includes a first pleated wall with a collection end and a second pleated wall that converges with the first pleated wall at the collection end. The first pleated wall and second pleated are both composed of a material that is porous and configured as a barrier to red blood cells. The washed blood solution is flowed on the pleated filter to separate the red blood cells from the rejuvenating solution and the wash solution. The separated red blood cells are then collected at the collection end.

An advantage of the present invention is that it provides a filter that can separate red blood cells from rejuvenating solution and plasma at high volumes.

Another advantage is that the present invention provides a gentle separation technique that is not complicated.

Yet another advantage is that the present invention can separate the red blood cells in a single fluid pass without the need for a pump.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a perspective view of a blood separation device according to the present invention;

FIG. 2 is a plan view of a pleated wall shown in FIG. 1 in a stretched out state;

FIG. 3 is a perspective view of another embodiment of a blood separation device according to the present invention;

FIG. 4 is a sectional view of the blood separation device shown in FIG. 1, taken along line 4-4; and

FIG. 5 is a flow chart diagram of a method of collecting red blood cells according to the present invention.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

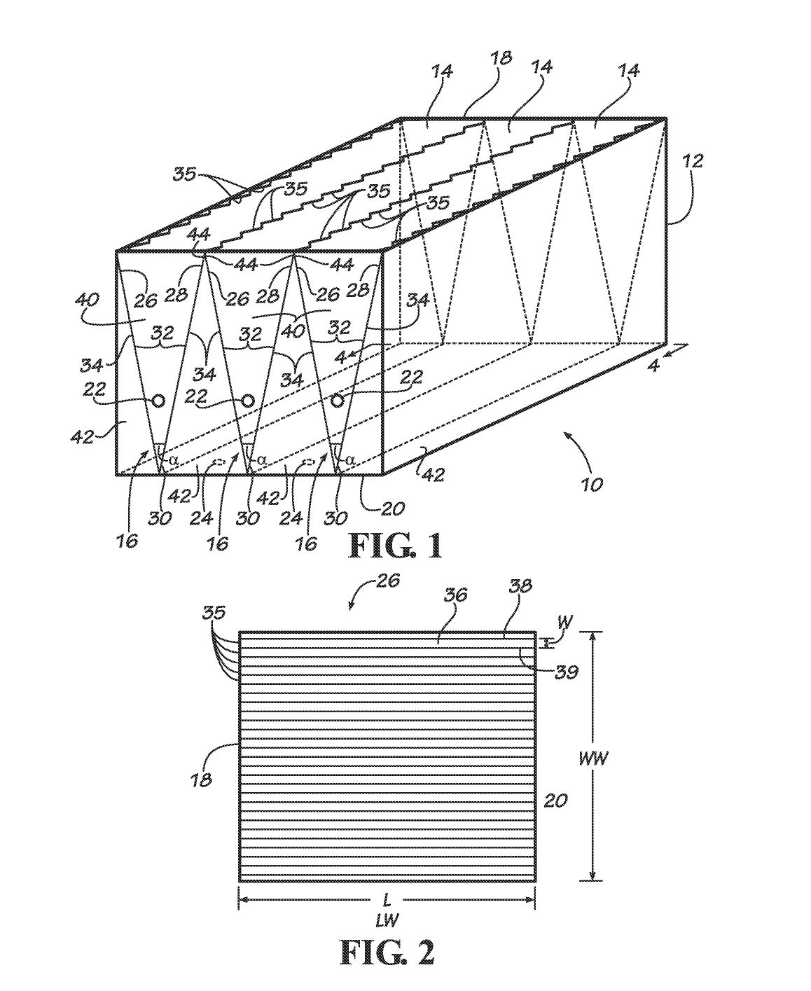

Referring now to the drawings, and more particularly to FIG. 1, there is shown a blood separation device 10 which generally includes a vessel 12 with blood inlets 14 and pleated filters 16 placed within the vessel 12. As can be seen, the vessel 12 has a top 18 that is open to form the blood inlets 14 that will flow a mixture containing red blood cells (RBCs) over the pleated filters 16. Although the top 18 is shown as being open, the top 18 could also partially cover the vessel 12 or completely cover the vessel 12 except for an opening(s) which would be the blood inlet(s). A bottom 20 of the vessel 12 can be closed, allowing for the blood separation device 10 to have multiple chambers formed within, which will be described below. The vessel 12 can also have multiple outlets 22, 24 formed that allow for the removal of separated components from the blood separation device 10. The vessel 12 is shown as having a rectangular prism shape, but the shape of the vessel 12 could be adjusted as desired to form differently shaped blood separation devices 10. It is useful for the vessel 12 to be made out of a biocompatible material that is non-toxic to RBCs, to provide maximum RBC viability following separation.

Multiple pleated filters 16 are placed within the vessel 12. For ease of reference, the remaining description will focus mostly on one pleated filter but it should be understood that either one or multiple pleated filters 16 could be included in the blood separation device 10 of the present invention. The pleated filter 16 can include a first pleated wall 26 and a second pleated wall 28. The pleated walls 26, 28 can converge toward the bottom 20 of the vessel 12 at a collection end 30 of the pleated filter 16, forming a V-shaped pleated filter 16. When the pleated walls 26, 28 form a V-shaped pleated filter 16, they will have an angle of convergence a relative to each other at the bottom 20 of the vessel 12. The angle of convergence a is contemplated as any angle that can form the V-shaped pleated filter 16, but it has been found that small values, between approximately 0.1 to 2.5 degrees, for the angle a are useful values to construct the pleated filter 16. FIG. 1 shows an angle of convergence a that is significantly larger than 0.1 to 2.5 degrees for illustrative purposes, and it should be understood that the angle of convergence a is contemplated as encompassing a wide range of values. The pleated walls 26, 28 each have a first surface 32 and a second surface 34 that is opposed to the first surface 32. The first surfaces 32 and second surfaces 34 are shown without texture for ease of illustration, but it should be understood that the first surfaces 32 and second surfaces 34 are pleated surfaces. The first surfaces 32 face each other and will be the surfaces of the pleated walls 26, 28 that first come into contact with the blood mixture when it is flowed across the pleated filter 16. FIG. 1 shows the pleated walls 26, 28 spanning the entire length of the pleated filter 16, but it is also contemplated that the pleated filter 16 could have two converging walls that include both pleated and unpleated sections.

The pleated walls 26, 28 are composed of a material that acts as a barrier to the RBCs and prevents the RBCs from passing through the first surface 32 while allowing the passage of other components such as blood plasma, rejuvenating solutions and wash solutions, to completely pass through the pleated walls 26, 28. Since RBCs are relatively large compared to the other components of blood mixtures, the pleated walls 26, 28 can be composed of a porous material that has pores sized to be smaller than the RBCs but larger than the other components of the blood mixture. This allows for the RBCs to be retained on the first surface 32 of the pleated walls 26, 28, while allowing for other components of the blood mixture to pass through the pleated filter 16. The average diameter of healthy RBCs is approximately 5-10 microns, so a material with pores that have a diameter of approximately 4 microns can act as an effective barrier to the RBCs. The material can also have unique surface features such as charge and chemical additives that prevent RBCs from passing through the first surface 32. Materials that can be used to create the pleated walls 26, 28 include filter papers and membranes. One known material that can be used is commercially sold by the Pall Corporation under the trade name of CytoSep® membrane. Other materials that have similar functional properties can also be used to form the pleated walls 26, 28.

Referring now to FIG. 2, pleated wall 26 is shown in a stretched out state. As can be seen, the pleated wall 26 has many pleats 35 formed on the surface of the pleated wall 26. Each pleat 35 can be defined as a pleat valley 36 between a pair of pleat boundaries 38, 39. The pleats 35 have a length L defined by the length of the pleated valley 36 and a width W defined by the distance between one pleated boundary 38 of the pleat 35 to the other pleated boundary 39 of the pleat 35. When the pleated wall 26 is part of the pleated filter 16 in the blood separation device 10, the pleat length L can extend from the top 18 of the vessel 12 to the bottom 20 and will define a wall length LW of the pleated filter 16. FIG. 2 shows the pleats 35 as having length L that is perpendicular to the bottom 20 of the vessel 12, but the pleats 35 could also have a length L that forms an acute angle relative to the bottom 20. The length L of the pleats 35 can be adjusted to give a longer or shorter pleated wall 26, as desired. The width W of the pleats 35 determine how many pleats 35 can be included in the pleated wall 26. The pleated wall 26 has a wall width WW which will be equivalent to the width W of each pleat 35 added together. If each pleat 35 has the same width W, then the number of pleats 35 that can be included in the pleated wall 26 will be equivalent to the wall width WW divided by the width W of the pleats 35. When the pleated wall 26 is unstretched, its width will decrease and the pleats 35 can be angled relative to each other rather than being planar. Pleated wall 28 can be arranged identically or similarly to pleated wall 26.

When a blood mixture is flowed across the pleated filter 16, gravity and/or fluid pressure can force the blood mixture into contact with the material of the pleated wall 26 on the first surface 32. Any molecules in the blood mixture that are permeable through the material of the pleated wall 26 can diffuse through the first surface 32 and be drawn out of the pleated wall 26 to the second surface 34, and then drop off the second surface 34 due to gravity and/or fluid pressure. As described previously, the material of the pleated wall 26 is configured to prevent the passage of RBCs through the first surface 32, causing them to accumulate along the first surface 32 toward the collection end 30. By adding pleats 35 to the pleated wall 26, the surface area of the pleated filter 16 can be dramatically increased without increasing the wall width WW or wall length LW. This allows for a significantly larger first surface 32 to come into contact with the blood mixture that flows through the blood separation device 10, increasing the volume of blood mixture that can have the RBCs separated out along the first surface 32. For example, when the vessel 12 has a volume of 250 mL, the pleated filters 16 can be pleated to produce first surfaces 32 with a total surface area of between approximately 0.5 to 1.0 square meters throughout the blood separation device 10. The combined surface area of the first surfaces 32 that can fit in the vessel 12 depends on the arrangement of the pleats 35 on the first surface 32 and the number of pleated filters 16 that can fit in the vessel 12. To increase the combined surface area of the first surfaces 32, a greater number of pleats 35 can be added to the first pleated wall 26 and second pleated wall 28 or a greater number of pleated filters 16 can be arranged in the vessel 12.

As can be seen in FIGS. 1 and 4, the pleated filter 16 separates the vessel 12 into an inlet chamber 40 that is fluidly connected to the blood inlet 14 and has a boundary defined by the first surfaces 32 of pleated walls 26, 28 and a filtrate chamber 42 which is defined by a space between the second surfaces 34 of the pleated walls 26, 28 and the bottom 20 of the vessel 12. When multiple pleated filters 16 are utilized in the blood separation device 10, multiple inlet chambers 40 and filtrate chambers 42 can be formed, as shown in FIGS. 1 and 4. As blood mixture flows into the inlet chamber 40, the RBCs in the blood mixture will stay in the inlet chamber 40 while other components of the blood mixture that are permeable through the pleated walls 26, 28 can collect into the filtrate chamber 42. As shown in FIG. 1, the pleated walls 26, 28 converge toward the collection end 30 of the pleated filter 16, which is where separated RBCs will accumulate. An outlet 22 can be formed in the vessel 12 near the collection end 30 and in fluid communication with the inlet chamber 40 to remove the separated RBCs from the inlet chamber 40. The outlet 22 can be attached to a collection container (not shown) that will hold the separated RBCs. The RBCs can be drawn through the outlet 22 by the force exerted on the RBCs during the separation process, a vacuum in the outlet, and/or a rinse along the first surface 32 to flow the RBCs into the collection bag. The outlet 22 can be placed close to the bottom 20 of the vessel 12, or could be placed a vertical distance from the bottom 20 to reduce the risk of pulling other separated blood mixture components from the filtrate chamber 42 into the inlet chamber 40 and re-mixing them with the separated RBCs. While an outlet 22 in the vessel 12 is shown to remove the separated RBCs from the blood separation device 10, it is also contemplated that the pleated filter 16 can be removed from the vessel 12 after separation and the RBCs can be collected from the pleated filter 16 outside of the vessel 12. An outlet 24 can also be formed through the vessel 12 in fluid communication with the filtrate chamber 42 to remove other blood mixture components that are collected in the filtrate chamber 42. It is useful to have the outlet 24 formed through the bottom 20 of the vessel 12, so that the filtrate chamber 42 does not get an accumulation of separated other blood mixture components that could possibly diffuse through the pleated filter 16 back into the inlet chamber 40 and re-mix with the separated RBCs. While the pleated filter 16 is shown as separating blood components inside vessel 12 as part of blood separation device 10, the pleated filter 16 could be independently used in a different configuration to separate blood components without straying from the scope of the present invention.

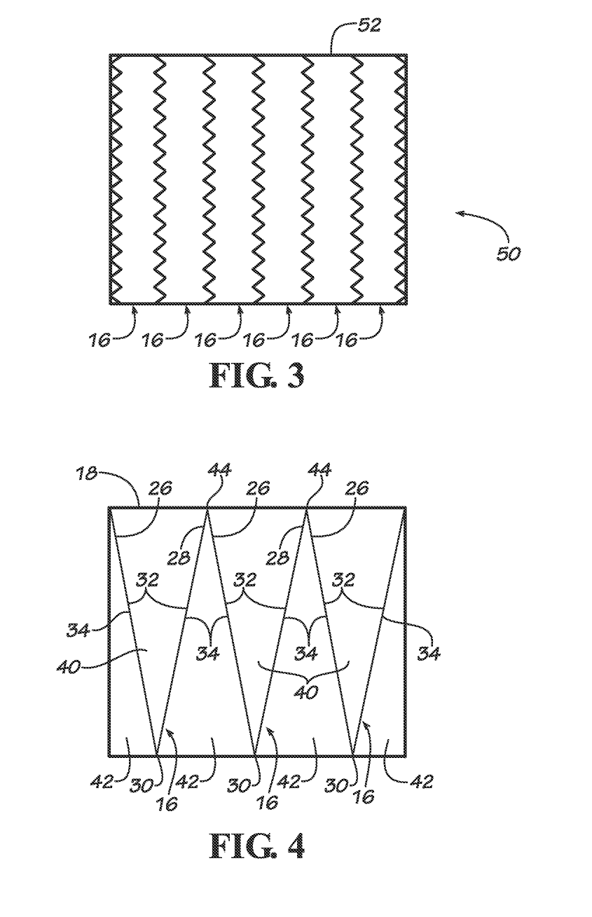

The pleated walls 26, 28 can have a joining end 44 that is opposite the collection end 30 and adjacent the top 18 of the vessel 12. As shown in FIGS. 1 and 4, the joining ends 44 of two adjacent pleated walls 26, 28 can be joined together to create a blood separation device 10 with multiple pleated filters 16. FIG. 1 illustrates a blood separation device 10 with three pleated filters 16, but as can be seen in FIG. 3 that number can be increased. FIG. 3 shows a top view of a blood separation device 50 that has six pleated filters 16 included in a vessel 52. By increasing the number of pleated filters 16 in the blood separation device, the effective filtration surface area, which corresponds to the area of the first surfaces 32, of the blood separation device is increased which allows for a greater volume of blood component mixture to be separated in the device. The pleated filters 16 could all be separable filters that are connected at joining ends 44, or could be formed from a single sheet of filter paper or a single membrane. It is contemplated that ten or more pleated filters 16 could be included in a blood separation device, if desired.

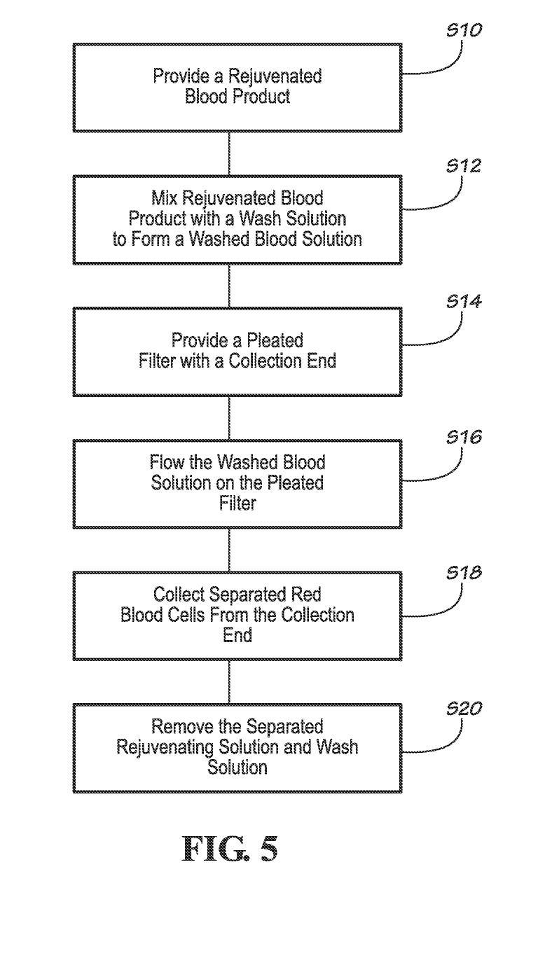

Referring now to FIG. 5, a method of collecting red blood cells is illustrated in a flow chart diagram. A rejuvenated blood product is provided that includes a mixture of RBCs and a rejuvenating solution (S10). The rejuvenating solution can be any solution that is able to keep the RBCs viable during storage and transport, with rejuvesol® Solution being an effective rejuvenating solution of sodium pyruvate, inosine, adenine, dibasic sodium phosphate and monobasic sodium phosphate. Optionally, the RBCs can be incubated with the rejuvenating solution prior to collection. The rejuvenated blood product is mixed with a wash solution to form a washed blood solution (S12). The wash solution can be any solution that can act as a solvent for the rejuvenating solution, and is preferably permeable through the pleated filter 16 previously described. The wash solution can be a mixture of medical grade saline solution and glucose, which is widely available, or a different wash solution. The previously described pleated filter 16 is provided (S14) and the washed blood solution is flowed on the pleated filter 16 to separate the RBCs from the rejuvenating solution and wash solution (S16). If desired, step S16 can be repeated as many times as necessary to sufficiently separate the rejuvenating solution and wash solution from the RBCs. After the RBCs have been separated from the wash solution and rejuvenating solution, they can then be collected (S18) from the collection end 30 of the pleated filter 16. The separated rejuvenating solution and wash solution can also be removed (S20) concurrently with or before the collection of the separated RBCs to minimize the possibility that the separated rejuvenating solution or wash solution will be mixed back in with the RBCs.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A filter for separating blood components, comprising:
a first pleated wall having a collection end; and
a second pleated wall converging with said first pleated wall at said collection end, wherein said first pleated wall and said second pleated wall are each composed of a material that is porous and configured as a barrier to red blood cells.

2. The filter according to claim 1, wherein said first pleated wall and said second pleated wall each include a pleated surface having a plurality of pleats formed thereon.

3. The filter according to claim 2, wherein each of said plurality of pleats has a length that extends in a direction towards said collection end.

4. The filter according to claim 1, wherein said first pleated wall and said second pleated wall form an angle of convergence relative to each other at said collection end.

5. The filter according to claim 4, wherein said angle of convergence is between approximately 0.1 to 2.5 degrees.

6. The filter according to claim 1, wherein said second wall has a joining end opposite said collection end.

7. The filter according to claim 6, further including a third pleated wall connected to said joining end, said third pleated wall being porous and configured as a barrier to red blood cells.

8. The filter according to claim 6, wherein said filter has a top and a bottom, said joining end defining said top and said collecting end defining said bottom.

9. The filter according to claim 1, wherein said material is permeable to at least one of saline solution, glucose, sodium pyruvate, inosine, adenine, dibasic sodium phosphate and monobasic sodium phosphate.

10. The filter according to claim 8, wherein said material is at least one of a filter paper and a membrane.

11. The filter according to claim 1, wherein said first pleated wall and said second pleated wall converge to form a V-shaped filter.

12. A blood separation device, comprising:
a vessel;
a blood inlet formed in said vessel; and
a pleated filter placed within said vessel, said pleated filter separating said vessel into an inlet chamber fluidly connected to said blood inlet and a filtrate chamber, said pleated filter being porous and configured as a red blood cell barrier between said inlet chamber and said filtrate chamber.

13. The blood separation device according to claim 11, further including a first outlet fluidly connected to said inlet chamber and a second outlet fluidly connected to said filtrate chamber.

14. The blood separation device according to claim 13, wherein said pleated filter includes a first pleated wall and a second pleated wall, said first pleated wall and said second pleated wall converging adjacent to said first outlet.

15. The blood separation device according to claim 14, wherein said material is permeable to at least one of plasma, saline solution, glucose, sodium pyruvate, inosine, adenine, dibasic sodium phosphate and monobasic sodium phosphate.

16. The blood separation device according to claim 14, wherein said vessel has a bottom and said first pleated wall and said second pleated wall form an angle of convergence relative to each other at said bottom.

17. The blood separation device according claim 16, wherein said angle of convergence is between approximately 0.1 to 2.5 degrees.

18. The blood separation device according to claim 16, wherein said first pleated wall and said second pleated wall each have a plurality of pleats formed thereon, each of said plurality of pleats having a length that extends in a direction toward said bottom.

19. A method of collecting red blood cells, comprising:
providing a rejuvenated blood product including red blood cells and a rejuvenating solution;
mixing said rejuvenated blood product with a wash solution to form a washed blood solution;
providing a pleated filter including a first pleated wall with a collection end and a second pleated wall that converges with said first pleated wall at said collection end, said first pleated wall and said second pleated wall being composed of a material that is porous and configured as a barrier to red blood cells;
flowing said washed blood solution on said pleated filter to separate said red blood cells from said rejuvenating solution and said wash solution; and
collecting said separated red blood cells at said collection end.

20. The method according to claim 19, further including the step of removing said separated rejuvenating solution and said separated wash solution from said pleated filter.
